SEQUENCE LISTING (2 sequences)

SEQ ID NO: 1 - protein, 293 aa, synthetic construct [sequence omitted]

SEQ ID NO: 2 - protein, 299 aa, synthetic construct
MADDAGAAGGPGGPGGPGMGNRGGFRGGFGSGIRGRGRGRGRGRGRGRGARGGKAEDKEWMPVTKLGRLVKDMKIKSLEEIYLFSLPIKESEIIDFFLGASLKDEVLKIMPVQKQTRAGQRTRFKAFVAIGDYNGHVGLGVKCSKEVATAIRGAIILAKLSIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFTDHLVKTHTRVSVQRTQAPAVATTHHHHHH

(19) United States
(12) Patent Application Publication
(10) Pub. No.: US 2025/0333446 A1
(43) Pub. Date: Oct. 30, 2025

(54) DIETARY SUPPLEMENTS, COMPOSITIONS, AND USES THEREOF FOR INCREASING TELOMERASE ACTIVITY AND EXTENDING TELOMERE LENGTH

(71) Applicant: IMAGINE PHARMA LLC, PITTSBURGH, PA (US)

(72) Inventors: NGOC THAI, PITTSBURGH, PA (US); JONATHAN POLLETT, PITTSBURGH, PA (US)

(73) Assignee: IMAGINE PHARMA LLC, PITTSBURGH, PA (US)

(21) Appl. No.: 19/092,807

(22) Filed: Mar. 27, 2025

Related U.S. Application Data
(60) Provisional application No. 63/571,675, filed on Mar. 29, 2024.

(51) Int. Cl. C07K 14/00; A61K 9/00; A61K 38/00

(52) U.S. Cl. CPC C07K 14/001; A61K 9/0053; A61K 38/00

(57) ABSTRACT

Disclosed herein are dietary supplements, and nutraceutical compositions thereof, comprising a polypeptide having at least 80%, 85%, 90%, 95%, 98% or 99% sequence identity to the amino acid sequence set forth in SEQ ID NO 1 and 2; and methods of increasing telomerase activity and extending the length of telomeres using a composition comprising a polypeptide having at least 80%, 85%, 90%, 95%, 98% or 99% sequence identity to the amino acid sequence set forth in SEQ ID NO 1 or 2. Also disclosed is an isolated polypeptide according to SEQ ID NO 2.

### RELATED APPLICATION

This application claims priority to and the benefit of U.S. Provisional Patent Application No. 63/571,675, filed on Mar. 29, 2024, the contents of which are incorporated by reference.

### SEQUENCE LISTING

This application includes a Sequence Listing submitted in electronic format in accordance with the requirements of 37 C.F.R. §§ 1.834-1.835 and WIPO Standard ST.26. The Sequence Listing is provided as an XML file named “IMG021.PCT.xml,” created on Mar. 27, 2025, having a file size of 3,000 bytes. The Sequence Listing contains amino acid sequences that are integral to the invention disclosed herein. The Sequence Listing is hereby incorporated by reference in its entirety into the present application. The sequences are identified by sequence identifiers (SEQ ID NOs.) as referenced throughout the specification.

### BACKGROUND

It is widely known that dietary and nutritional supplements can play a role in prevention, alleviation, or delay of onset of many diseases or symptoms, can help boost the immune system, and may even play a pivotal role in the treatment of some diseases. Dietary and nutritional supplements may also play a role in helping to delay or prevent aging and the signs of aging.

As we age, we amass a series of maladies and conditions that affect our day to day lives. Age-related diseases are illnesses and conditions that occur more frequently in older people. Age is a significant risk factor in such conditions. Some of most common aging-associated diseases are atherosclerosis and cardiovascular disease, cancer, arthritis, cataracts, osteoporosis, type 2 diabetes, hypertension, and Alzheimer's disease. About 60% of the older populations suffer from obesity which leads to type 2 diabetes. Many also suffer from metabolic syndrome which increases risk factor for developing cardiovascular diseases. Out of the 150,000 people that die each day across the globe, about two thirds die of age-related causes. During ageing, the functionality of cells declines, and various types of cell-intrinsic damage and alterations in the niche and the circulating blood have been demonstrated to contribute to this age-related decline. Telomere, a region of repetitive nucleotide sequences (TTAGGG) associated with specialized proteins at the ends of linear chromosomes, length shortens with age. Progressive shortening of telomeres leads to senescence, apoptosis, or oncogenic transformation of somatic cells, affecting the health and lifespan of an individual. Shorter telomeres have been associated with increased incidence of age-related diseases, maladies, and mortality. Therefore, a telomeres length is an important factor in maintaining the life span of cells in various tissues. The introduction of “youthful” progenitor cells into the human body may help rejuvenate existing cells, increase telomere length, and allow the body to age more gracefully, and even reverse some effects of the aging process.

Imagine Pharma's U.S. Pat. No. 10,548,941 to Thai et al. (“Thai”) discloses and claims pharmaceutical compositions including a 40s ribosomal protein S2 (with 293 amino acids (see Sequence ID No. 1) and referred to as “IMG-1”) useful for the treatment of hypertension and hyperglycemia using a therapeutically effective amount of IMG-1. Thai identifies oral formulations that include 50-150 μg/kg of IMG-1, which for a 70-kg human amounts to 3.5 mg to 10.5 mg. Thai does not disclose use of IMG-1 for increasing telomerase activity and/or increasing telomere length.

### SUMMARY

Telomerase is an enzyme protein that catalyzes the addition of a telomere repeat sequence to the 3′ end of a telomere and serves to restore telomere loss due to cellular senescence. Telomerase is not expressed in most normal adult somatic cells, and telomere length is gradually decreased in cell replication.

In specific diseases, the telomere's end is abnormally rapidly lost and leads to aging or degeneration of immature cells. In the case of a human, cells expressing human telomerase (hTERT, human-telomerase reverse transcriptase) are found to bypass a normal cellular senescence pathway. When the expression of telomerase is induced in aged cells having a short telomere, a telomere length is increased, and phenotypes similar to younger cells are restored.

Since the loss of the telomeric repeat sequence is reduced by an increase in telomerase activity, it is expected that the induction of telomerase activity having an effect of adding a telomeric repeat sequence at the end of the telomere restarts the replication and division of somatic cells in a senescent or death phase and induces restoration of damaged tissue in which a large quantity of the senescent or death-phase cells are distributed. Since telomere length has a role in many aging-related diseases, increasing telomere length could provide therapeutic benefits to alleviate, or reverse, age-related diseases.

In one aspect of the disclosure, a composition is disclosed comprising a polypeptide having at least 80%, 85%, 90%, 95%, 98% or 99% sequence identity to the amino acid sequence set for the in SEQ ID NO(s) 1-2 (or fragments thereof), when administered to somatic cells in culture (in vitro; ex vivo) and to a mammal (in vivo) causes an increase in telomerase activity in somatic cells, and a corresponding increase in telomere length.

In one aspect, the peptide disclosed herein may include one or more peptides having at least 80%, 85%, 90, 95%, 96%, 97%, 98% or 99% sequence homology to SEQ ID No(s) 1-2.

In another aspect, the peptide disclosed herein may include a peptide having a difference in 1 or more amino acids, 2 or more amino acids, 3 or more amino acids, 4 or more amino acids, 5 or more amino acids, 6 or more amino acids, or 7 or more amino acids from the peptide of SEQ ID No(s) 1-20r fragments thereof.

In yet another aspect, the disclosure provides a composition comprising a polypeptide with an amino acid sequence having at least 80% sequence homology with the amino acid sequence according to SEQ ID No(s) 1-2, or a fragment thereof together with one or more nutritional additives, that when administered to a human causes an increase in telomerase activity and increase in length of telomeres.

A dietary supplement comprising an amino acid sequence according to SEQ ID No(s) 1 or 2 is provided. The dietary supplement may be formulated in a dosage form selected from the group consisting of a capsule, a cachet, a pill, a tablet, a powder, a granule, a pellet, a bead, a particle, a troche, a lozenge, a gel, a liquid, a suspension, a solution, an elixir, and a syrup. The dietary supplement formulation may further include one or more additional components, including but not limited to a carrier, an excipient, a binder, an antioxidant, a colorant, a flavoring agent, a preservative, a buffer, a diluent, a second food additive or supplement, and/or combinations thereof.

### DETAILED DESCRIPTION OF THE DRAWINGS

The compositions and methods disclosed herein are further described by the accompanying figures shown in the Appendix, attached hereto.

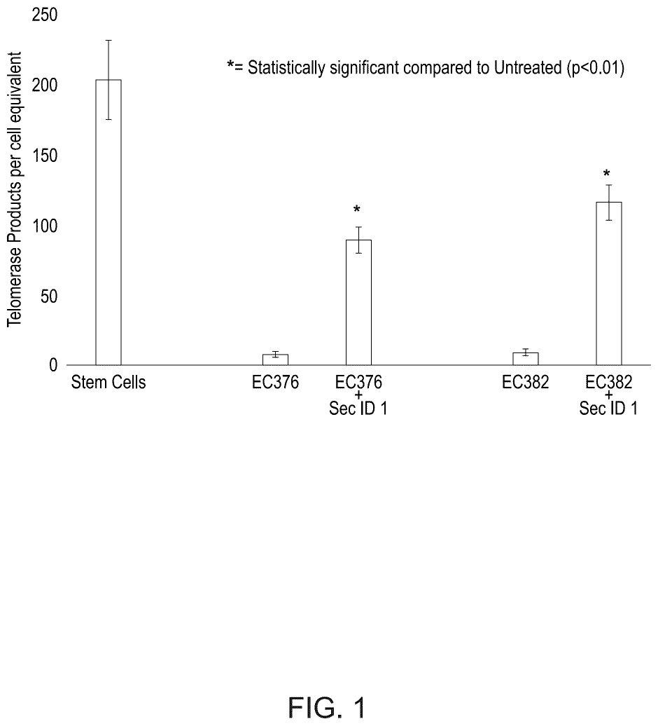

FIG. 1 shows use of SEQ ID 1 causes an increase in telomerase activity in endothelial cells in vitro. Endothelial cells were treated with IMG-1 (20 μg/mL, SEQ ID 1) for 72 hrs and then telomerase products per cell equivalent was measured by TeloTAGGG™ Telomerase PCR ELISAPLUS (Roche). EC376 refers to and EC 382 refers to two primary endothelial cell populations. Stem cells with established telomerase activity were used as a control. Telomerase activity was determined by telomerase products per cell equivalent.

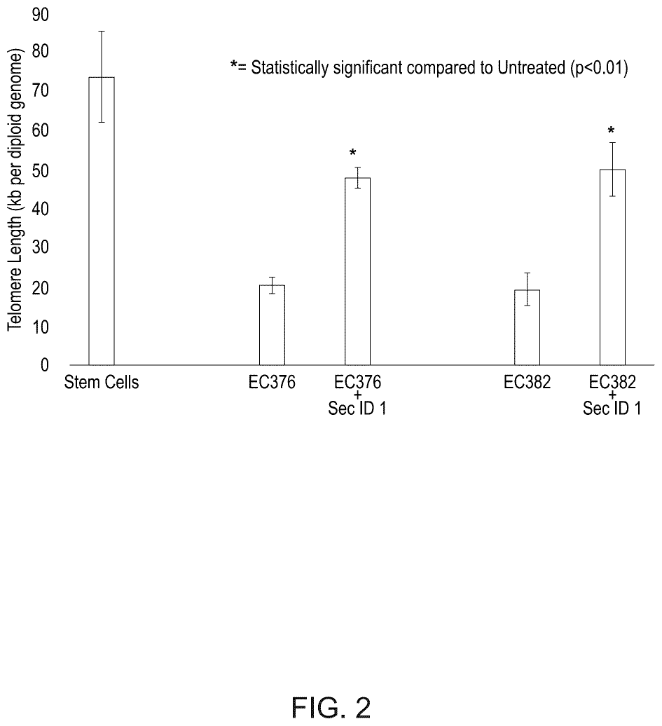

FIG. 2 shows use of SEQ ID 1 causes an increase in telomere length in endothelial cells in vitro. Using the same cells under the same conditions as described in FIG. 1, Telomere length was also measured 72 hrs post-treatment. Telomere length was determined using the Absolute Human Telomere Length Quantification qPCR Assay Kit (ScienCell Research Laboratories) as per manufacturer's instructions.

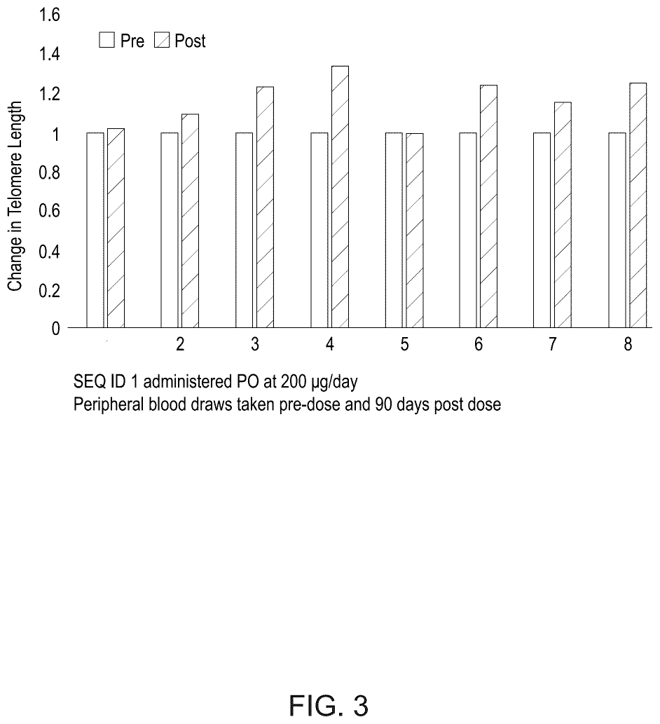

FIG. 3 shows use of SEQ ID 1 causes an increase in telomere length in vivo as seen by the analysis of blood samples taken from eight adult human subjects administered an oral dose (200 μg/daily) of the composition for 12-weeks. Telomere activity was determined by TeloTAGGG™ Telomerase PCR ELISAPLUS (Roche).

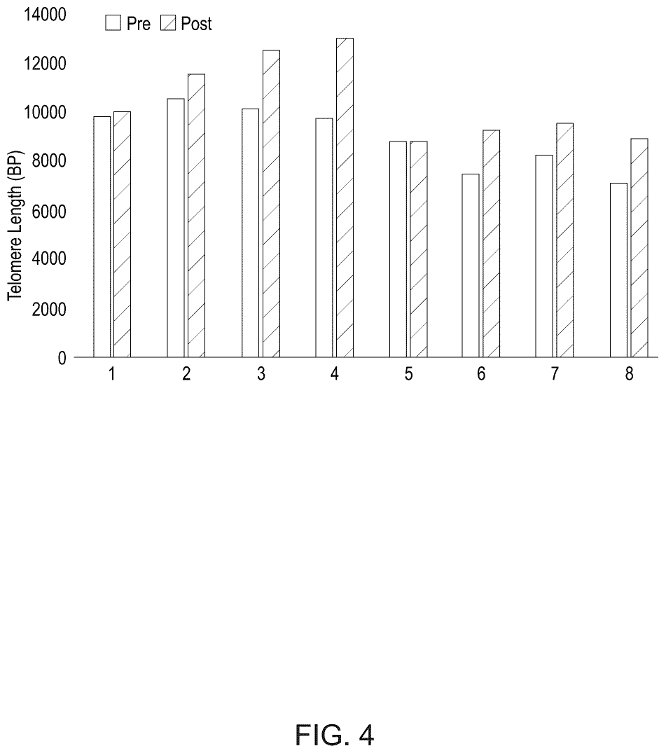

FIG. 4 shows use of SEQ ID 1 causes an increase in telomere length in vivo—as seen by the analysis of blood samples taken from adult human subjects pre and post administration of an oral dose in a range of 200 mg/ml of the composition. Telomere length was quantified as Kb per diploid genome and as base pairs.

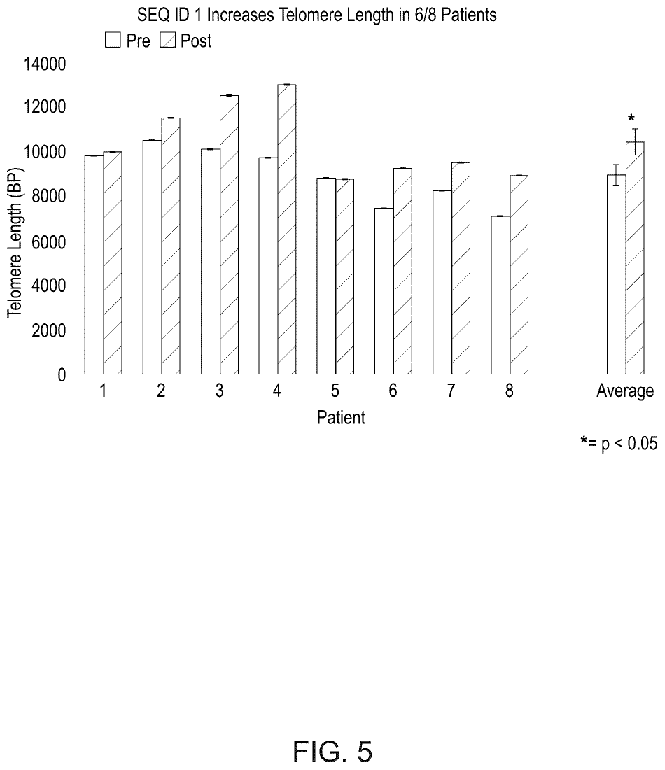

FIG. 5 shows use of SEQ ID 1 causes an increase in telomere length in vivo as seen by the analysis of blood samples taken from eight adult human subjects administered an oral dose (200 μg/daily) of the composition for 12-weeks. The average length in base pairs was 8963.875 (pre) and 10425 (post dosing of SEQ ID 1).

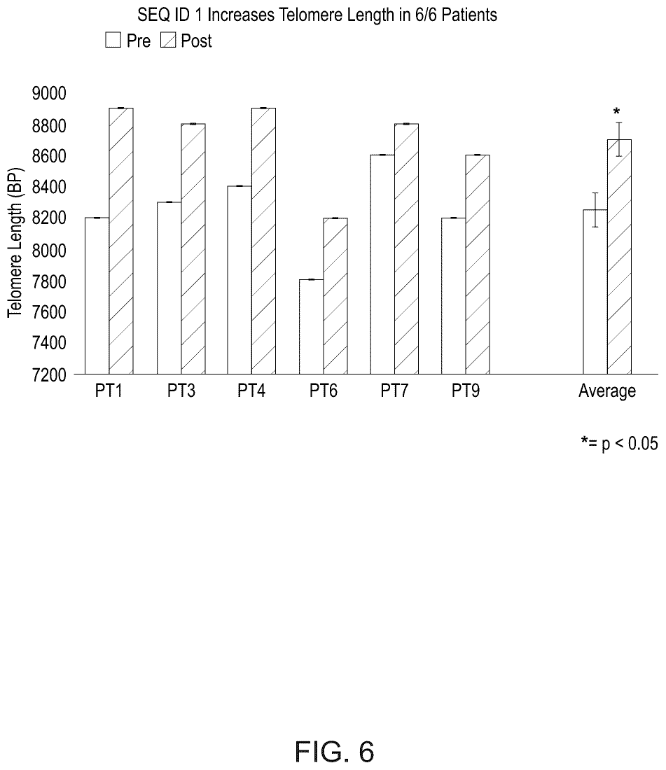

FIG. 6 shows use of SEQ ID 1 causes an increase in telomere length in vivo as seen by the analysis of blood samples taken from eight adult human subjects administered an oral dose (200 μg/daily) of the composition for 12-weeks. The average length in base pairs was 8250 (pre) and 8700 (post).

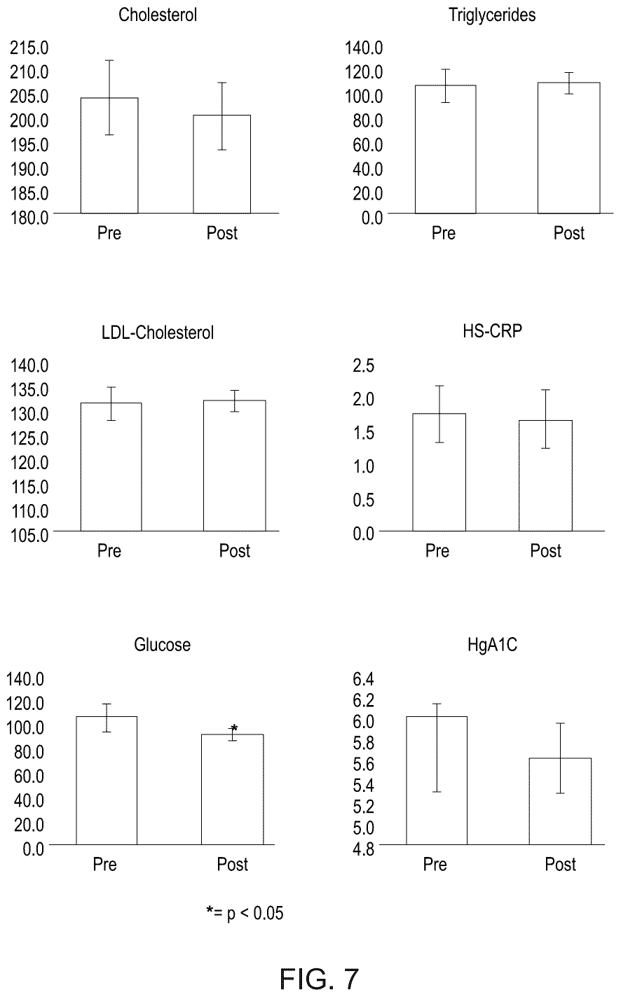

FIG. 7 shows test results in the observational study. Data shown is the aggregate average of participants pre and post dosing of SEQ ID 1 over 12 weeks. Cholesterol, Triglycerides, LDL and glucose are measured as mg/dL; Hs-CRP is measured as mg/L; and HgA1c is measured as percentage.

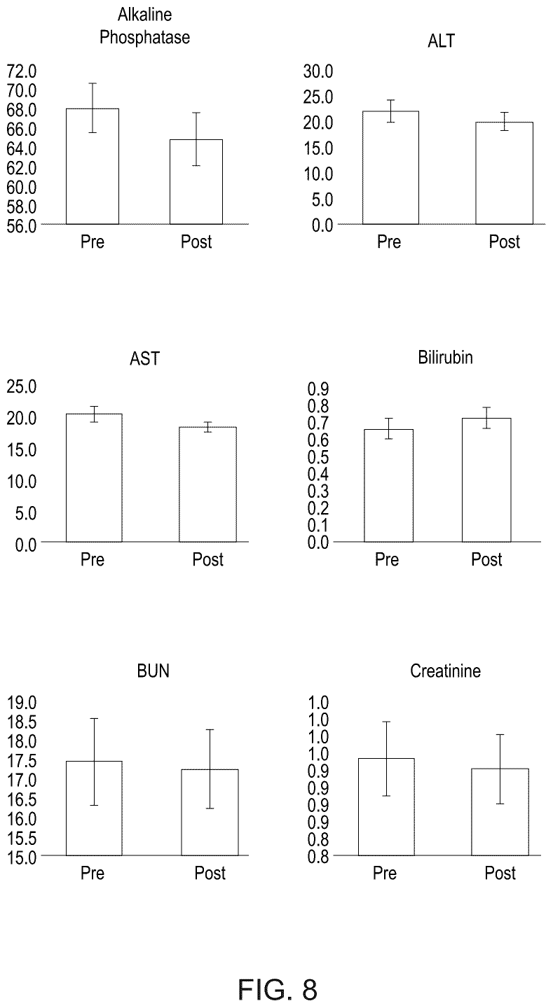

FIG. 8 shows the markers of liver function in study participants taking an oral dose of SEQ ID 1, pre and post study (data shown is the aggregate average of the participants, pre and post dosing of SEQ ID 1). Alkaline phosphatase, ALT and AST are measured as Units/L; and Bilirubin, BUN and Creatinine are measured as mg/dL.

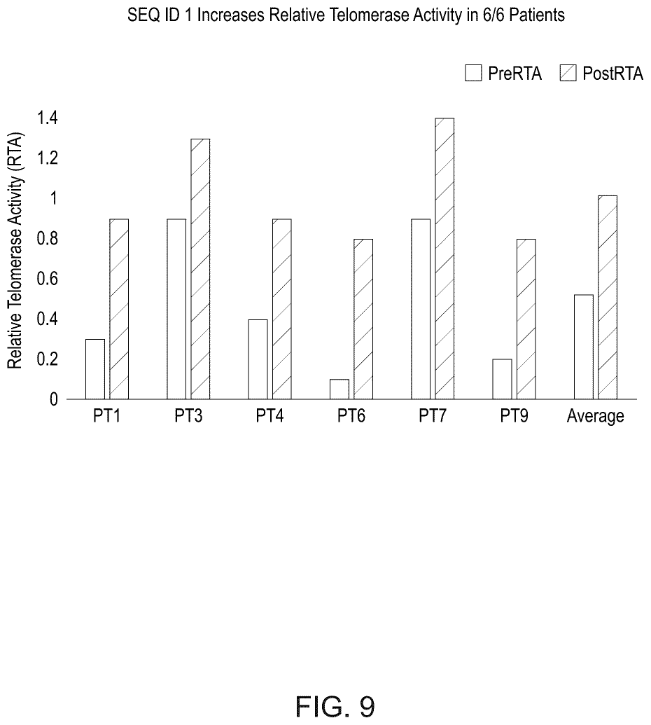

FIG. 9 shows that use of SEQ ID 1 causes an increase in telomerase activity in vivo as seen by the analysis of blood samples taken from six adult human subjects administered an oral dose (200 μg/daily) of the composition for 12-weeks. The average increase in telomerase activity (measured as Relative Telomerase Activity) was 0.494.

### DETAILED DESCRIPTION

The following terms are used in this disclosure to describe different embodiments. These terms are used for explanation purposes only and are not intended to limit the scope for any aspect of the subject matter claimed herein.

As used herein “GRAS” means an acronym for the phrase Generally Recognized As Safe, under sections 201 (s) and 409 of the Federal Food, Drug, and Cosmetic Act (the Act).

As used herein, “food” means a product containing a peptide, amino acid, protein, carbohydrate and/or fat and is intended for consumption and includes a source of nourishment and nutritional value. For example, the food may include, but is not limited to, a hot or cold cereal, a baking product, a beverage, pasta, bread, snack products, dairy products, and the like.

As used herein, “food product” means any ingredient, component or composition that provides nutritional value to a mammal, including humans.

As used herein, “isolated” or “purified” refers to a polypeptide that has been removed from at least one naturally associated component. For example, the polypeptide may be at least 1% pure, such as at least 5% pure, at least 10% pure, at least 20% pure, at least 40% pure, at least 60% pure, at least 80% pure, Pure, at least 90% pure, or at least 95% pure.

As used herein a “nutraceutical” is any functional food (including beverages) that provides an additional benefit other than its nutritional benefit.

As used herein “SEQ ID NO 1 or SEQ ID NO 2, etc.” refers to a protein, polypeptide, peptide fragment, or analogue thereof, and including any modification thereto, having an amino acid sequence having at least 85%, 90%, 95%, 98% or 99% sequence identity to the amino acid sequence according to SEQ ID No(s) 1 and/or 2 (See Table 1). Also contemplated is a peptide fragment, or analogue thereof, and including any modification thereto, having an amino acid sequence having at least 85%, 90%, 95%, 98% or 99% sequence identity to the amino acid sequence according to Table 1. For example, a fragment of SEQ. ID NO 1 may include Thai's sequence ID numbers 2-5, where Thai's subject matter is disclosed herein by reference. Likewise, a fragment of SEQ. ID NO 2 may include Thai's sequence ID numbers 2-5 that include a histidine tag comprised of 2-10 histidine moieties (e.g., 4-10 histidine moieties, 6-10 histidine moieties, or 6 histidine moieties) located at the N- or C-terminus of the particular peptide.

It has been demonstrated by in vitro studies using polypeptides according to SEQ ID NO 1 and SEQ ID NO 2 that treatment of myriad cell types (keratinocytes, enterocytes, islet, endothelial and pneumocyte cells) with polypeptides according to SEQ ID NO 1 or SEQ ID NO 2 added to cell culture medium causes stimulation and increase in cell growth, resulting in viable progenitor cells, as measured by the percent of CD133 positive cells in culture. It has also been shown that treatment of cells with a peptide according to SEQ ID NO 1 or SEQ ID NO 2 causes an increase in the anti-aging-related genes Sirts 1, 2 and 6, as well as a dramatic increase in mTor expression. The cells treated with a peptide according to SEQ ID 1 or 2 can be regenerated and propagated into the billions.

As used herein, the term “sequence identity” refers to the identity between two or more amino acid sequences expressed in terms of the identity or similarity between the sequences. Sequence identity can be measured in terms of percentage identity; the higher the percentage, the more identical the sequences are. The percentage identity is calculated over the entire length of the sequence. Homologs or orthologs of amino acid sequences possess a relatively high degree of sequence identity when aligned using standard methods. This homology is more significant when the orthologous proteins are derived from species which are more closely related (e.g., human and mouse sequences), compared to species more distantly related (e.g., human and C. elegans sequences). Methods of alignment of sequences for comparison are well known in the art. Various programs and alignment algorithms are described in: Smith & Waterman; Needleman & Wunsch, J. Mol. Biol. 48:443, 1970; Pearson & Lipman, Proc. Nat. Acad Sci. USA 85:2444, 1988; Higgins & Sharp, Gene, 73:23744, 1988; Higgins & Sharp, CABIOS 5:151-3, 1989; Carpet et al., Nuc. Acids Res. 16:10881-90, 1988; Huang et al. Computer Appls. in the Biosciences 8, 155-65, 1992; and Pearson et al., Meth Mol. Bio. 24:307-31, 1994. Altschul et al., J. Mol. Biol. 215:403-10, 1990, presents a detailed consideration of sequence alignment methods and homology calculations. The level of sequence identity may be determined using the NCBI Basic Local Alignment Search Tool (BLAST) (Altschul et al., J. Mol. Biol. 215:403-10, 1990), which is available from several sources, including the National Center for Biological Information (NCBI, National Library of Medicine, Building 38A, Room 8N805, Bethesda, Md. 20894, US) and on the Internet.

As used herein, “supplement” or “dietary supplement” (used interchangeably) means a composition that is intended to supplement the diet by providing certain nutrients or other molecules in contrast to a large number of calories, and as further defined by Dietary Supplement Health and Education Act (often referred to as DSHEA). Supplements may be in solid, semi-solid, or liquid form (e.g., including tablets, capsules, soft gels, gel caps, powders, bars, gummies, and liquids) to provide the polypeptides of the present invention. In addition to the polypeptides of the present invention, the supplements may contain any one or more of the following: vitamins, minerals, herbs, amino acids, essential fatty acids and other substances that are traditionally supposed to be supplements or which may be used in supplements or others.

It will be understood that a numerical value may be associated with a certain amount of experimental error. Thus, recitation of the qualifier “about” (or “approximately”) prior to a numerical error is meant to embody the experimental error that may be associated with the recited numerical value. To the extent that a numerical value obtained experimentally is not preceded by the expression “about” (or “approximately”) does not mean that the numerical value is not associated with a certain amount of experimental error.

TABLE 1
SEQ ID NOs 1 and 2

| | |
|---|---|
| SEQ ID | MADDAGAAGGPGGPGGPGMGNRGGFRGGFGSGIRGRGRG |
| NO. 01 | RGRGRGRGRGARGGKAEDKEWMPVTKLGRLVKDMKIKSL |
| (293 aa) | EEIYLFSLPIKESEIIDFFLGASLKDEVLKIMPVQKQTR |
| | AGQRTRFKAFVAIGDYNGHVGLGVKCSKEVATAIRGAII |
| | LAKLSIVPVRRGYWGNKIGKPHTVPCKVTGRCGSVLVRL |
| | IPAPRGTGIVSAPVPKKLLMMAGIDDCYTSARGCTATLG |
| | NFAKATFDAISKTYSYLTPDLWKETVFTKSPYQEFTDHL |
| | VKTHTRVSVQRTQAPAVATT |
| SEQ ID | MADDAGAAGGPGGPGGPGMGNRGGFRGGFGSGIRGRGRG |
| NO. 2: | RGRGRGRGRGARGGKAEDKEWMPVTKLGRLVKDMKIKSL |
| (299 aa) | EEIYLFSLPIKESEIIDFFLGASLKDEVLKIMPVQKQTR |
| | AGQRTRFKAFVAIGDYNGHVGLGVKCSKEVATAIRGAII |
| | LAKLSIVPVRRGYWGNKIGKPHTVPCKVTGRCGSVLVRL |
| | IPAPRGTGIVSAPVPKKLLMMAGIDDCYTSARGCTATLG |
| | NFAKATFDAISKTYSYLTPDLWKETVFTKSPYQEFTDHL |
| | VKTHTRVSVQRTQAPAVATTHHHHHH |

Amino acid residues may be post-translationally modified or conjugated with other functional or non-functional molecular groups. See, e.g., Guo et al. Mol. Biosyst. 7 (7): 2286-2295, 2011, describing generally antagonistic citrullination and methylation of human ribosomal protein S2 (e.g., SEQ ID NO. 1). Naturally, such modified amino acid residues are included in the amino acid sequences and within the scope of the compositions described herein. Additionally, selected amino acids may be substituted conservatively for alternative amino acids. For instance, valine (V) may be substituted with isoleucine (I) and vice versa, glutamic acid (E) may be substituted with aspartic acid (D) and vice versa, serine(S) may be substituted with threonine (T) and vice versa, phenylalanine (F) may be substituted with tyrosine (Y) and vice versa, and arginine (R) may be substituted with lysine (K) and vice versa.

The polypeptides and/or polypeptide fragments according to, for example, SEQ ID NO(s) as disclosed in Table 1 may be produced under conditions known in the art for protein production, such as production in bacteria, yeast or by synthetic means.

### Formulations

Further details on techniques for formulation and administration may be found in the latest editions of REMINGTON'S PHARMACEUTICAL SCIENCES (Mack Publishing Co., Easton, Pa.); REMINGTON: THE SCIENCE AND PRACTICE OF PHARMACY, Alfonso R. Gennaro, editor, 20th ed. Lippingcott Williams & Wilkins: Philadelphia, Pa., 2000; and PHARMACEUTICS: THE SCIENCE OF DOSAGE FORM DESIGN, Aulton, M. E., editor, 2nd Edition, Churchill Livingstone Publishing, 2002.

Suitable formulations of compositions comprising SEQ ID NO 1 or SEQ ID NO 2 include tablets, capsules, solutions, suspensions, powders. Sublingual delivery systems include, but are not limited to, dissolvable tabs under and on the tongue, liquid drops, and beverages.

For oral administration, a compound, or a salt, hydrate, or solvate thereof, may be further combined with one or more solid peptides for the preparation of tablets, capsules, pills, powders, granules, or other suitable dosage forms. For example, SEQ ID NO 1 or SEQ ID NO 2 may be combined with at least one excipient selected from the group consisting of fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

For a liquid or gel capsule, licap, or tablet formulation, a dose of SEQ ID NO 1 or SEQ ID NO 2 ranging from 0.05 mg to 1 mg per capsule/tablet is provided, and an exemplary dose of SEQ ID 1 or 2 of 0.2 mg to 0.4 mg per capsule/tablet.

For a liquid formulation, a dose of SEQ ID NO 1 or SEQ ID NO 2 ranging from 0.05 mg/mL to 1 mg/ml (e.g., 0.1 mg/ml or 0.2 mg/mL or 0.4 mg/mL), and an exemplary dose of SEQ ID NO 1 or SEQ ID NO 2 of 0.2 mg/mL or 0.4 mg/ml (e.g., volume of 1 mL or volume of 2 mL).

For a finely divided solid or powder, a dose of SEQ ID NO 1 or SEQ ID NO 2 ranging 0.05 mg to 0.1 mg (e.g., 0.2 mg or 0.4 mg) as a powder is provided.

Liquid preparations include solutions, suspensions, and emulsions, for example, water or water-propylene glycol solutions. The compositions and formulations may take such forms as suspensions, solutions, or emulsions in oily or aqueous vehicles, and may contain formulation agents such as suspending, stabilizing, and/or by dispersing agents. Alternatively, the peptide may be in powder form, obtained by aseptic isolation of sterile solid or by lyophilization from solution, for reconstitution with a suitable vehicle, e.g., sterile, pyrogen-free water, before use.

Aqueous solutions suitable for oral use can be prepared by dissolving the SEQ ID NO 1 or SEQ ID NO 2 polypeptide components in water; and optionally adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Solid dietary supplements and nutritional compositions of SEQ ID NO 1 or SEQ ID NO 2 for oral administration may optionally contain, one or more nutritional composition ingredients or compounds, and one or more of the following: carrier materials such as corn starch, gelatin, acacia, microcrystalline cellulose, kaolin, dicalcium phosphate, calcium carbonate, sodium chloride, alginic acid, and the like; disintegrators including microcrystalline cellulose, alginic acid, and the like; binders including acacia, methylcellulose, sodium carboxymethylcellulose, polyvinylpyrrolidone, hydroxypropylmethylcellulose, ethylcellulose, and the like; and lubricants such as magnesium stearate, stearic acid, silicon fluid, talc, waxes, oils, colloidal silica, and the like. The usefulness of such excipients is well known in the art.

Liquid dietary supplements or nutritional compositions of SEQ ID NO 1 or SEQ ID NO 2 for oral administration can be prepared in water or other aqueous vehicles. In addition to the above enumerated ingredients or compounds, liquid nutritional compositions can include suspending agents, wetting agents, sweeteners, and coloring and flavoring agents.

Various liquid and powder nutritional compositions can be prepared by conventional methods. Various ready-to-drink formulations (“RTDs”) are contemplated.

Dietary supplements and nutritional formulations of SEQ ID NO 1 or SEQ ID NO 2 may also comprise the peptide and at least one or more of a probiotic; vitamin, such as vitamin C or vitamin E, beta carotene; minerals such as zinc, manganese, ferrous sulfate, or calcium; or one or more nutritional additives.

The SEQ ID NO 1 or SEQ ID NO 2 peptides in such formulations may comprise from 1% by weight to 99% by weight, or alternatively, 0.1% by weight to 99.9% by weight. “Nutraceutically acceptable carrier” and “pharmaceutically acceptable carrier” mean any carrier, diluent, additive or excipient that is compatible with the other ingredients of the formulation and not deleterious to the user. In accordance with one embodiment, suitable nutraceutically acceptable carriers can include ethanol, aqueous ethanol mixtures, water, fruit, and/or vegetable juices, and combinations thereof.

In one embodiment, a dietary supplement or nutraceutical composition is a fine powder composition for reconstitution in water and comprises a polypeptide having at least an 80% sequence identity to the amino acid sequence according to SEQ ID NO 1 or SEQ ID NO 2, or fragments thereof, and optionally sodium citrate, and optionally sucralose.

In one embodiment, a dietary supplement or nutraceutical composition is a capsule and comprises a polypeptide having at least an 80% sequence identity to the amino acid sequence according to SEQ ID NO 1 or SEQ ID NO 2, or fragments thereof, and one or more of microcrystalline cellulose, and hydroxypropyl methylcellulose, and the like.

The methods described herein may be further understood in connection with the following non-limiting examples, which are offered by way of illustration and not by way of limitation with respect to subject matter claimed herein.

### EXAMPLES

### Example 1

### A Composition Comprising a Polypeptide According to SEQ ID 1 Causes an Increase in Telomere Length and Telomerase Activity In Vitro.

Primary (human) endothelial cells were treated with a composition comprising a peptide having a sequence identity according to SEQ ID 1 at a concentration of 10-20 μg/mL, although a range of 2 μg/mL up to 30 μg/mL may be used. At seven (7) days post treatment, cells were collected and assayed for telomerase activity and telomere length was measured using a TeloTAGGG™ Telomere Length Assay and TeloTAGGG™ Telomerase PCR ELISA kit, Millipore Sigma. The results are quantitatively assessed and presented as either relative telomere length (RTL) or average telomere length in base pairs. This assay is widely applied in aging research, cancer biology, genetic studies, and disease research, providing a reliable and accurate means of measuring telomere length in biological samples.

Telomere activity was determined by TeloTAGGG™ Telomerase PCR ELISAPLUS (Roche) and Telomere length was determined using the Absolute Human Telomere Length Quantification qPCR Assay Kit (ScienCell Research Laboratories) as per manufacturer's instructions. In brief, cells were resuspended in 2×10⁵ cells per 200 μl Lysis reagent and incubated on ice for 30 minutes. Then centrifuged at 16,000×g for 20 minutes at 4° C. To set up the PCR reaction 5 μg of cellular protein was used. Following PCR 2.5 μl of the amplification product was added to 2 tubes containing 10 μl of denaturation reagent and incubated at 25° C. for 10 minutes. Following incubation one tube received 100 μl hybridization buffer T and one tube 100 μl hybridization buffer IS and 100 μl of the mixture was transferred to the precoated microplate and incubated at 37° C. on a shaker for 2 hrs. The plate was then washed 3× with 250 μl washing buffer and following the final wash 100 μl Anti-DIG-HRP working solution was added to each well and incubated at 25° C. for 30 minutes on a shaker. Following incubation, the plate was washed 5× with 250 μl washing buffer, and 100 μl TMB substrate solution was added to each well and the plate incubated for an addition 20 minutes at 25° C. on a shaker. Following incubation, 100 μl of stop reagent was added and the absorbance of the samples was read at 450 nm using a reference wavelength of 690 nm.

Primary endothelial cells treated with a composition comprising a peptide according to SEQ ID 1 were shown to increase telomere length by (on average) 29 kilo bases per diploid genome (an increase of over 246%). See FIG. 1

Treatment was also shown to increase the telomerase activity in cells dramatically: from on average 8.65 telomerase products per cell equivalent to over 100 telomerase products per cell equivalent (an increase of over 11×). See FIG. 2

Treatment was also shown to increase the number of progenitor cells (as a measure of CD133 production, data not shown) from 4.3% to over 11%.

Preliminary microarray analysis of cells exposed to a composition comprising a peptide according to SEQ ID 1 were also shown to increase expression of the anti-aging-related genes Sirts 1, 2 and 6, as well as a dramatic increase in mTor expression. Microarray analysis was performed by mRNA-seq carried out using Illumina next generation sequencing (NGS) platform, a high-throughput method that enables the analysis of gene expression by sequencing RNA molecules to quantify and profile their levels. This technique provides comprehensive data on the transcriptome for understanding gene activity and expression patterns.

### Example 2

### A Composition Comprising a Polypeptide According to SEQ ID 1 Causes an Increase in Telomere Length In Vivo.

A 90-day human clinical observation study was conducted to investigate the safety and efficacy of SEQ ID 1 (dose 200 μg/day in a 1 mL solution of H₂O by perorally (PO)) as a wellness and longevity supplement with 21 adult participants (aged 45 and older), including those with metabolic syndrome, diabetes, and individuals with known diagnoses. Participants consumed a polypeptide according to SEQ ID NO: 1 mixed in sterile water at an oral dose in the range of 100-200 μg/day (perorally (“PO”)). SEQ ID 1 was isolated in PBS but diluted to a usable concentration in H₂O. SEQ ID 1 was consumed daily each morning for 12 weeks. Blood samples were collected from participants prior to administration and at 90 days (12 weeks) post administration.

Quantitative laboratory biomarkers (telomere length, lipid profile, hs CRP, fasting glucose and HbA1c), a questionnaire (Multiple System Questionnaire), and clinical assessment were performed on this cohort before and at 90 days after initiation of oral SEQ ID 1 (200 μg/day in a 1 mL solution of H₂O). The study expanded to include a broader qualitative cohort and participants were given oral SEQ ID 1 and reported monthly their perception of symptomatic changes with SEQ ID 1.

Telomere length was measured in the blood samples collected, pre and post administration. The data shows that telomere length increased (measured as kb per diploid genome, and as base pairs) following oral administration of a composition comprising a polypeptide according to SEQ ID NO 1.

Telomere length was determined using the Absolute Human Telomere Length Quantification qPCR Assay Kit (ScienCell Research Laboratories) as per manufacturer's instructions. Genomic DNA was first extracted from the biological samples, such as blood, tissue, or cultured cells, using standard DNA isolation protocols. The concentration of the extracted DNA was then quantified to ensure adequate amounts for the assay. The qPCR reaction mix was prepared by combining the DNA sample with telomere-specific primers, primers for a reference gene (such as 36B4), a reaction buffer, dNTPs, and a DNA polymerase enzyme. The reaction was performed in a qPCR machine, which amplified both the telomere region and the reference gene in parallel. Real-time monitoring of DNA amplification was achieved by detecting fluorescent signals emitted during each cycle of amplification, with the cycle threshold (Ct) value corresponding to the amount of DNA. The telomere length was then determined by comparing the Ct values of the telomere amplification to the Ct values of the reference gene amplification. The relative telomere length (RTL) was calculated as the ratio of telomere amplification to reference gene amplification, and standard curve analysis was used to convert the RTL to an absolute telomere length, measured in base pairs. The final results were expressed as the mean of multiple replicate measurements. This method provides an accurate and reliable means of quantifying telomere length in human DNA, making it suitable for studies related to aging, cell proliferation, and disease states.

Telomerase activity was measured in peripheral blood mononuclear cells from 8 participants and were analyzed pre and post consumption (PO) of SEQ ID 1. To obtain the genomic DNA to determine telomere length the Monarch® Genomic DNA Purification Kit (New England Biolabs) was used. In short, 100 μl of blood was transferred to a 1.5 ml microfuge tube and 10 μl Proteinase K, 3 μl RNase A and 100 μl of Blood Lysis Buffer was added and the mixture vortexed and incubated for 5 minutes at 56° C. in a heating block and vortexed occasionally. Following incubation 400 μl gDNA Binding Buffer was added to the sample and mixed by pulse-vortexing for 5-10 seconds. The lysate was transferred to a gDNA Purification Column pre-inserted into a collection tube and centrifuged for 3 minutes at 1,000×g and then for 1 minute at maximum speed and the flow through discarded and the column was added to a new collection tube and washed 2× with 500 μl gDNA Wash Buffer, inverted 5 times, and centrifuged for 1 minute at maximum speed. Following washing the gDNA Purification Column was put in a DNase-free 1.5 ml microfuge tube and 100 μl preheated (60° C.) gDNA Elution Buffer was added and incubated at room temperature for 1 minute. The genomic DNA was eluted in 100 μl preheated elution buffer (10 mM Tris-Cl, pH 9.0, 0.1 mM EDTA) and centrifuged for 1 minute at maximum speed to elute the gDNA. Telomerase activity was measured using a TeloTAGGG™ Telomerase PCR ELISA kit, (available from Millipore Sigma, or Roche), per manufacturers instructions. The results are quantitatively assessed and presented as relative telomere length (RTL).

In brief, blood cells were resuspended in 2×10⁵ cells per 200 μl Lysis reagent and incubated on ice for 30 minutes. Then centrifuged at 16,000×g for 20 minutes at 4° C. To set up the PCR reaction 5 μg of cellular protein was used. Following PCR 2.5 μl of the amplification product was added to 2 tubes containing 10 μl of denaturation reagent and incubated at 25° C. for 10 minutes. Following incubation one tube received 100 μl hybridization buffer T and one tube 100 μl hybridization buffer IS and 100 μl of the mixture was transferred to the precoated microplate and incubated at 37° C. on a shaker for 2 hrs. The plate was then washed 3× with 250 μl washing buffer and following the final wash 100 μl Anti-DIG-HRP working solution was added to each well and incubated at 25° C. for 30 minutes on a shaker. Following incubation, the plate was washed 5× with 250 μl washing buffer, and 100 μl TMB substrate solution was added to each well and the plate incubated for an addition 20 minutes at 25° C. on a shaker. Following incubation, 100 μl of stop reagent was added and the absorbance of the samples was read at 450 nm using a reference wavelength of 690 nm.

Of the 8 participants, 6 showed detectable telomerase enzyme activity. See FIG. 9. The t-test for the difference in the telomerase activity was p=2.54547E-05 (which would be characterized as extremely significant). The average between the untreated and the treated was 0.52 vs 1.02 RTA (Relative Telomerase Activity) a difference of 0.5 (more specifically 0.494).

Notably all 6 with detectable telomerase activity, also showed decreased high sensitivity C-reactive protein (hs-CRP) levels; whereases the 2 participants without detectable telomerase activity had no improvement, or a slight increase in, hs-CRP activity. PT1 (Participant “1”) had a 0.2 decrease in hs-CRP (measured in mg/dL) from 0.9 to 0.7 (pre and post SEQ ID 1), PT2 had an increase in hs-CRP of 0.9, from 1.2 to 2.1 (pre and post SEQ ID 1); PT3 had a 0.4 decrease in hs-CRP from 1.2 to 0.8 (pre and post); PT4 had a 0.1 decrease in hs-CRP from 0.4 to 0.3 (pre and post); PT5 had an increase in hs-CRP of 0.2, from 0.5 to 0.7 (pre and post); PT6 had a 0.9 decrease in hs-CRP, from 2.0 to 1.1 (pre and post); and PT7 had a 4.6 decrease in hs-CRP, from 8.5 to 3.9 (pre and post); and PT9 had a 0.2 decrease in hs-CRP from 2.0 to 1.8 (pre and post).

No negative side-effects were reported by the participants. Twenty participants reported positive improvement in overall wellbeing, 18 reported increased energy and feeling better, 12 reported less inflammation and joint pain, 6 had improved bowel habits and cessation of irritable bowel syndrome symptoms.

Among participants with cholesterol in the normal range at baseline (less than 200 mg/dL) and who consumed the oral dose of SEQ ID 1:9 of 15 participants saw lower total cholesterol (60%) (measured as mg/dL); 8 of 15 participants had lower LDL cholesterol (53%); 6 of 15 participants had increased HDL (40%) (measured as mg/dL); and 5 of 15 participants had lowered triglycerides (33%) measured as mg/dL. See FIG. 7, which shows the average values pre and post study. For those participants with an elevated cholesterol level (greater than 200 mg/dL), 5 of 6 participants saw a reduction in cholesterol (83%).

Following 90 days of oral doses of SEQ ID 1:11 of 15 (73%) of participants saw a reduced level of high-sensitivity C-reactive protein (hs-CRP) measured as mg/L; and among the participants with an initially elevated C-reactive protein level (greater than 1.0 at the onset of the study), 6 of 7 saw an improvement in their C-reactive protein score by the end of the study. In addition, 13 of 14 (93%) of participants saw a decrease in fasting blood glucose (measured as mg/dL) at 90 days, and 6 of 15 (40%) saw a reduction in HbA1c levels (measured as a percentage). See FIG. 7, which shows the average values (N=21) pre and post study.

Across all participants, markers of liver function (bilirubin (mg/dL), AST (U/L), ALT (U/L), alkaline phosphatase (U/L)) and renal function (BUN (mg/dL), creatinine (mg/dL)) remained stable and within normal clinical ranges before and after the day PO dose of SEQ ID 1. See FIG. 8, which shows the average values (N=21) pre and post study.

Both the quantitative and qualitative cohorts demonstrated that SEQ ID 1 is very safe for human consumption. Liver and kidney biomarkers, longstanding standard for examining toxicity, were unchanged from their normal values before and after SEQ ID 1 therapy in the quantitative group. As further supporting evidence, there were no reported side effects in the 38 participants in the qualitative cohort. In the qualitative cohort, 37 reported increased energy, 19 reported improved joint pain, 7 reported improved blood glucose levels, lower insulin and HgNA1c, 7 reported a reduction in irritable bowel syndrome symptoms, 3 reported an improved cholesterol level, and 2 reported an improvement in hot flashes. Data was collected from participants via monthly telephonic interviews.

Moreover, SEQ ID 1 appeared to demonstrate beneficial effects as measured by laboratory values listed above. Improvements in total cholesterol, LDL cholesterol, hs CRP and fasting blood glucose were seen in a majority of participants (>50%), notably with lower fasting blood glucose seen in 93% and inflammation reduction (hs CRP) in 73% of the participants. A minority of the 15 quantitative cohorts (<50%) exhibited improvements in triglycerides, HDL, HbA1C. Triglyceride improvement was seen in 33% of the participants.

SEQ ID 1 administration (PO) decreases overall inflammation in 73% of participants; in this 15-participant cohort, after 90-days of SEQ ID 1 PO dosing, 11/15 (73%) participants had a reduction in hs-CRP suggesting lower body inflammation. Stratified to those with initial abnormal CRP (>1), 6/7 (86%) pts showed improvement in CRP.

Telomerase activity and telomere length studies performed on 8 participants showed activated telomerase activity and increased telomere length in peripheral blood of 6 (75%) after 90 days of SEQ ID 1PO dosing. These findings strongly suggest SEQ ID 1 possesses anti-aging and regenerative effects. Interestingly, there was strong correlation between increased telomere length and reduction of the inflammation marker, hs-CRP.

A Type 2 diabetes mellites study participant with elevated fasting blood glucose of 313, HgbA1c of 14.0, and elevated total cholesterol of 263 was administered a composition comprising SEQ ID 1 at 200 μg PO once daily (“QD”). After 3 months, blood tests showed improvement of HgbA1c reduced to 12.0, and fasting blood glucose was reduced to 145 mg/dL, and total cholesterol reduced by 44 points (263 reduced to 219). Hs-CRP also reduce by 0.9.

### Example 3

The Safety and Toxicity of SEQ ID NO 2 was Evaluated to Confirm that SEQ ID NO 2 is a Safe (within the Meaning of FD&C Act), Non-Toxic Food Product and/or Dietary Supplement.

The host organism used to produce SEQ ID NO 2 is a non-toxic and non-pathogenic host that has been demonstrated through scientific procedures to be safe and suitable for use in food production and is the production organism for numerous GRAS Notified ingredients, which have received No Questions Letters from US FDA. The safety of the SEQ ID NO 2 gene expressed in the production strain was established through confirmation of the absence of plasmids containing antibiotic resistance genes and maintenance of genomic stability throughout the production process.

Recognized guidelines used to evaluate the safety of microbial enzyme preparations for use in food (Pariza and Johnson, 2001) were applied to assess the safety of SEQ ID NO 2 produced by a modified strain of E. coli BL21 (DE3) (i.e., BL21-pVB-1A0C1-SEQ ID NO 2). A comprehensive review and study concluded that SEQ ID NO 2 produced by E. coli BL21 (DE3) [BL21-pVB-1A0C1-SEQ ID NO 2] is accepted as a safe and suitable food ingredient.

The safety of SEQ ID NO 2 was evaluated in a standard battery of in vitro and in vivo genetic toxicity assays as well as a repeated dose, sub chronic (90-day) oral toxicity study in rodents. SEQ ID NO 2 was confirmed as non-genotoxic and there were no adverse effects noted any dose level tested in the 90-day study (up to 2.5 mg/kg bw/day via oral gavage). Additionally, no adverse effects were reported in a 90-day human clinical study investigating the safety and efficacy of SEQ ID NO 2 in healthy subjects at an oral dose of 200 μg/day. These unpublished studies corroborate the safety of SEQ ID NO 2.

SEQ ID NO 2 is a peptide consisting of 299 amino acids with an approximate molecular weight of 35,000 Da and may be produced by fermentation of a genetically modified Escherichia coli strain (the BL21 (DE3) System), that was transduced with the vector pVB-1A0C1-SEQ ID NO 2 to generate BL21-pVB-1A0C1-SEQ ID NO 2 (Because E. coli does not possess the components required for E. coli pathogenicity, E. coli BL21 (DE3) and strains derived from DE3 are non-pathogenic.

The composition of the SEQ ID NO 2 peptide may be that of a free-flowing liquid, including phosphate-buffered saline (PBS) as a solvent. The E. coli BL21 (DE3) strain was purchased from Invitrogen. The E. coli BL21 (DE3) strain was developed for T7 RNA polymerase-based gene expression by introducing a lambda prophage containing a T7 RNA polymerase under the control of the lacUVA promoter. The integration of the lambda prophage generated a Gal-phenotype. The T7 RNA polymerase enables the high-level inducible expression of genes driven by the T7 promoter. The expression of the T7 RNA polymerase in E. coli BL21 (DE3) is usually achieved by the addition of 0.1-1 mM IPTG. The E. coli B strain has been used in the laboratory for almost 100 years and was developed for T7 RNA polymerase-based gene expression by DE3 prophage integration, UV treatment and selection. The pVB-1A0C1-SEQ ID NO 2 plasmid was transformed into E. coli BL21 (DE3) and the resultant production strain was designated as BL21-pVB-1A0C1-SEQ ID NO 2 was generated. BL21-pVB-1A0C1-SEQ ID NO 2 was grown on nutrient medium containing the antibiotic kanamycin. Kanamycin is used during fermentation to maintain stable growth of the recombinant strain during fermentation, and IPTG is used to induce expression of the RPS2 gene. The suitable use of IPTG and antibiotics during fermentation of enzyme preparations has been reviewed previously (GRN 126, GRN 485). The complete removal of culture broth by centrifugation and washing steps ensures that all the additives in the media are removed and not carried over to the next step of production process.

The BL21 (DE3) strain is widely used in the biopharmaceutical industry for the manufacture of recombinant pharmaceutical proteins and is the production organism for numerous food ingredients that have been the subject of GRAS Notifications that received No Questions Letters from US FDA (e.g., GRN 485, 571, 919, 922, and 925). Peptide purification using this strain is well documented.

### Formulation

After final recovery/purification, the final protein concentrate may be diluted to a final working concentration of 0.2 mg/mL with sterile potable water or PBS, with a final pH of 8-10. In one embodiment, packaging may be conducted at ≤26° C. The packaging material (food grade) consist of an inner bag of PET Pouch and an outer bag that contains 3 layers PET, PE/ALU/PET. The amount per package is 1 kg, each package is labeled with the following: name of product, weight, shelf-life/best before date, product code. The product is stored at ≤−18° C.

### Histidine Tag

A Histidine (His)-tag was cloned onto the carboxy end of SEQ ID 1. This DNA sequence specifying a string of six histidine residues is frequently used in vectors for production of recombinant proteins. Protein purification requires methods that are highly specific and robust, regardless of scale. Expressed His-tagged proteins can be purified easily because the string of histidine residues binds to several types of immobilized metal ions, including nickel, cobalt and copper, under specific buffer conditions. In addition, anti-His-tag antibodies are commercially available for use in assay methods involving His-tagged proteins. In either case, the tag provides a means of specifically purifying the recombinant protein without a protein-specific antibody or probe.

His-tags may allow for the selective extraction of a protein of interest from the cell lysate without modifying the protein's function or identity. The addition of His-tags, especially low count His-tags attached to the carboxy end of a protein, generally had no significant effect on the structure of the attached protein (Carson et al., 2007; Booth et al., 2018, Aslantas and Surmeli, 2019; Roshanak et al., 2023; Yan et al., 2023).

### Intended Use and Estimated Daily Intake:

SEQ ID NO 2 is intended for use as an ingredient in a broad range of beverages as well as a food additive (for example, in yogurt) at a maximum level of 1000 μg/serving/day. The intended use of SEQ ID NO 2 by food/beverage category is provided.

To calculate the theoretical estimated daily intake of SEQ ID NO 2 from all potential beverage or yogurt types to which SEQ ID NO 2 may be added, the maximum concentration levels of SEQ ID NO 2 in μg/mL (ppm) for each beverage category and for yogurt were calculated using FDA's reference amounts commonly consumed per eating occasion (RACC; 21 CFR 101.12) and assuming an SEQ ID NO 2 level not exceeding 1000 μg/serving (Tables 2-3).

TABLE 2
Intended uses of SEQ ID NO 2 in select foods and beverages

| | | | | Proposed | |
|---|---|---|---|---|---|
| | | | | Maximum Use | |
| | | | Typical | Level | |
| | | | Serving Size, | μg/ | |
| NHANES WWEIA Food Category and Code ^{a} | | | g or mL ^{b} | serving | μg/mL |
| Nonalcoholic | Fruit drinks | 7204 | 240 mL | 1000 | 4.17 |
| beverages | Fruit juice | 7002, | 240 mL | 1000 | 4.17 |
| | | 7004, 7006 | | | |
| | Vegetable juice | 7008 | 240 mL | 1000 | 4.17 |
| | Coffee | 7302 | 360 mL | 1000 | 2.78 |
| | Tea | 7304 | 360 mL | 1000 | 2.78 |
| | Soft drinks (regular and diet) | 7102, 7202 | 360 mL | 1000 | 2.78 |
| | Sport and energy drinks | 7206 | 360 mL | 1000 | 2.78 |
| | Diet sport and energy drinks | 7104 | 360 mL | 1000 | 2.78 |
| | Nutritional beverages | 7208 | 240 mL | 1000 | 4.17 |
| | Smoothies and grain drinks | 7220 | 240 mL | 1000 | 4.17 |
| | Water (tap or bottled) | 7702, 7704 | 360 mL | 1000 | 2.78 |
| | Flavored and/or carbonated | 7802 | 360 mL | 1000 | 2.78 |
| | water | | | | |
| | Enhanced water | 7804 | 360 mL | | 2.78 |
| | Milk (whole, reduced fat, | 1002, | 240 mL | 1000 | 4.17 |
| | low-fat, nonfat, Lactaid, | 1004, | | | |
| | buttermilk, kefir) | 1006, 1008 | | | |
| | Flavored milk (whole, | 1202, | 240 mL | 1000 | 4.17 |
| | reduced fat, low-fat, nonfat) | 1204, | | | |
| | | 1206, 1208 | | | |
| | Milk shakes and other dairy | 1402 | 240 mL | 1000 | 4.17 |
| | drinks | | | | |
| | Milk substitutes | 1404 | 240 mL | 1000 | 4.17 |
| Yogurt | Yogurt (regular and Greek) | 1820, 1822 | 170 g | 1000 | 5.88 |

^{a} Note that codes were not necessarily used in their entirety. The group code represents the group code from which individual food codes were taken.
^{b} Typical serving sizes derived from reference amounts commonly consumed per eating occasion (RACC; 21 CFR 101.12).

TABLE 3
Theoretical two-day average daily intake of SEQ ID NO 2 peptide
from the proposed uses by the U.S. population 2+ years
and select sub-populations, NHANES 2017- March 2020

| | | | | | 90^{th} | | 90^{th} |
|---|---|---|---|---|---|---|---|
| | Total | Users | | Mean | Percentile | Mean | Percentile |
| Population | sample, n | N^{a} | % | mg/day | | mg/kg bw/day | |
| U.S. 2+ y | 10,116 | 10,116 | 100 | 6.4 | 10.9 | 0.10 | 0.16 |
| Children 2-5 y | 979 | 979 | 100 | 3.5 | 5.2 | 0.19 | 0.31 |
| Children 6-12 y | 1,203 | 1,203 | 100 | 4.1 | 6.3 | 0.11 | 0.18 |
| Adolescents 13-18 y | 1,135 | 1,135 | 100 | 5.3 | 8.8 | 0.08 | 0.14 |
| Adults 19+ y | 6,799 | 6,799 | 100 | 7.0 | 11.4 | 0.09 | 0.14 |

^{an}Un-weighted number of users; % user, per capita and per user estimates were based on the National Health and Nutrition Examination Survey (NHANES) 2017-March 2020 using the statistical weights provided by the National Center for Health Statistics (NCHS).

The intended use of SEQ ID NO 2 is for addition of prepackaged product containing up to 1 mg (1000 μg) to one serving of one product per day. Thus, the above intake estimates purposely overestimate SEQ ID NO 2 exposure from all included food types and assumes SEQ ID NO 2 addition to all proposed food types.

### Toxicology

Because SEQ ID NO 2 is a protein with no evidence of previous human consumption, a standard battery of supportive in vitro and in vivo safety studies was performed for the SEQ ID NO 2 protein with purity of approximately 85%. Genotoxicity and oral dosing studies were performed.

Under the conditions of this studies performed, SEQ ID NO 2 at doses up to 200 mg/kg/day was found to be non-toxic and did not induce genotoxic micronucleus formation in immature murine erythrocytes of the mouse nor did it impact the percentage of CD71⁺ reticulocytes in circulation in the mice tested.

### 14-Day Oral Range-Finding Study

Prior to conducting a full, repeat-dose 90-day oral gavage study, a composition comprising SEQ ID NO 2 was evaluated in a non-GLP-compliant preliminary 14-day repeat-dose oral gavage range-finding study in Sprague Dawley CD® IGS rats. Forty healthy male (n=20) and female (n=20) rats were distributed into four groups (5 animals/sex/group) and doses of 0 (vehicle control, PBS), 0.025, 0.25, and 2.5 mg/kg bw/day of SEQ ID NO 2 were administered twice daily (7 days/week) via oral gavage. The intermediate and low dose levels were selected to derive a dose-response for any effects observed. The test article was mixed weight-to-volume (w/v) in PBS that served as the vehicle control. Fresh formulations containing 0.005, 0.05, and 0.5 mg/ml of SEQ ID NO 2 were prepared daily. The main study test design and dose levels are presented below in Table 4.

TABLE 4
Dose Levels in 14-Day Oral Range-Finding Study of SEQ ID NO 2

| | Animals/ | | | Dose | Oral Gavage Dose |
|---|---|---|---|---|---|
| | Group | Oral Gavage Dose | Dose Volume | Concentration | of Active Ingredient |
| Group | M/F | (mg/kg bw/day) | (mL/kg bw/day) | (mg/mL) | (mg/kg/day) |
| 1 | 5/5 | Vehicle Control | 5 | 0 | 0 |
| | | 0 | | | |
| 2 | 5/5 | SEQ ID NO 2 (Low) | | 0.005 | 0.0213 |
| | | 0.025 | | | |
| 3 | 5/5 | SEQ ID NO 2 | | 0.05 | 0.213 |
| | | (Intermediate) | | | |
| | | 0.25 | | | |
| 4 | 5/5 | SEQ ID NO 2 (High) | | 0.5 | 2.13 |
| | | 2.5 | | | |

bw = body weight; F = Female; M = Male.
^{an} Appropriate concentrations of the test article in vehicle to achieve the target dose level of active, based on 10 mg/mL stock solution of 85% active.
^{b} Phosphate-buffered saline (PBS, Manufacturer: Corning, Lot #: 26322010, and Exp. Date: Jan. 31, 2025).

As evidenced by Table 5, dose confirmation analysis indicated that the administered doses were within 92.5% of the target dose in the low-dose group, within ˜88% in the mid-dose group, and within 99.7% of the target dosage for the highest dose group. The test article was considered to be homogeneously distributed and met the target concentrations in the Group 2 (0.005 mg/mL), Group 3 (0.05 mg/mL), and Group 4 (0.5 mg/mL) mixtures. Based on the overall stability, homogeneity, and concentration verification results, the animals were considered to have received the targeted concentrations of SEQ ID NO 2.

TABLE 5
Dose formulation analysis results for concentration verification

| | | Dose Level | Measured Al | |
|---|---|---|---|---|
| Collection Day | Group | (mg/mL) | Conc. (mg/mL) | % of Target¹ |
| 1 | 1 | 0 | ND | NA |
| | 2 | 0.005 | 0.005 | 92.5 |
| | 3 | 0.05 | 0.044 | 87.9 |
| | 4 | 0.5 | 0.5 | 99.7 |

ND = Not Detected;
NA = Not Applicable
¹% of Target = [Measured dose (ppm)/Target Dose (ppm)] × 100.
² Samples were collected but not analyzed. The dose preparations were considered stable.

Animals were monitored for viability, signs of gross toxicity, and behavioral changes at least once daily during the study and were measured for body weight, average body weight gain, feed intake, and feed efficiency. Over the course of the study, there were no test article-related mortalities, clinical observations, or changes in body weight, body weight gain, or food consumption in male and female rats attributable to SEQ ID NO 2 administration. All males and females were active and healthy during the study. Mean body weights and daily body weight gain and mean food consumption for male and female rats in Groups 2-4 were comparable to control Group 1 throughout the study. Gross necropsy revealed blood clots in the abdomen of one control Group 1 male. This observation was considered incidental and not related to test article administration. It was concluded that under the conditions of the study, and based on the toxicological endpoints evaluated, there were no test article related changes attributed to the administration of SEQ ID NO 2. Therefore, based on the lack of adverse effects and the expected tolerability of doses up to 2.5 mg/kg/day in a study of longer duration, the same doses were used in the 90-day study.

### 90-Day Oral Toxicity Study in Rats

In a GLP-compliant 90-day repeat dose oral gavage study, 80 Sprague Dawley CD® IGS rats (10 animals/sex/group) were administered SEQ ID NO 2 at doses of 0 (vehicle control), 0.025, 0.25, and 2.5 mg/kg bw/day to evaluate potential sub chronic toxicity of SEQ ID NO 2. This study was conducted consistent with FDA's Redbook 2000, IV.C.4 a. Short-Term Toxicity Studies with Rodents (2007) and OECD Test Guideline 408: Health Effects, Repeated Dose 90-Day Oral Toxicity Study in Rodents (2018). An appropriate amount of the vehicle control or test substance was administered daily (7 days/week) via oral intubation to each rat at a rate of 5 mL/kg. The test substance was administered at concentrations of 0.005, 0.05, and 0.5 mg/ml. Table 6 summarizes aspects of the oral toxicity study.

TABLE 6
Dose levels in SEQ ID NO 2 90-day repeat dose oral toxicity study

| | | | Dose | Dose |
|---|---|---|---|---|
| | Animals/ | Oral Gavage Dose | Volume | Concentration |
| Group | Group M/F | (mg/kg bw/day) | (mL/kg bw/day) | (mg/mL) |
| 1 | 10/10 | Vehicle Control | 5 | 0 |
| | | 0 | | |
| 2 | 10/10 | SEQ ID NO 2(Low) | | 0.005 |
| | | 0.025 | | |
| 3 | 10/10 | SEQ ID NO 2 (Intermediate) | | 0.05 |
| | | 0.25 | | |
| 4 | 10/10 | SEQ ID NO 2 (High) | | 0.5 |
| | | 2.5 | | |

bw = body weight; F = Female; M = Male.
^{a} Appropriate concentrations of the test article in vehicle to achieve the target dose level of active, based on 10 mg/mL stock solution of 85% active.
^{b} Vehicle was PBS.

All animals were observed for viability, signs of gross toxicity, and behavioral changes at least once daily during the study and approximately weekly for a battery of detailed clinical observations. Animals were also measured/examined for standard safety endpoints, including, ophthalmologic evaluations, body weights/gains, feed consumption, hematology, coagulation, clinical chemistry, urinalysis, thyroid hormones, gross necropsy, organ weights, and histopathology (see test report for a full list of endpoints/organs evaluated). There were no significant changes to body weight parameters or mean food consumption in male and female rats attributable to test substance administration. One control Group 1 male was found dead on Day 91 due to improper dosing. Administration of SEQ ID NO 2 to male and female Sprague-Dawley rats by daily oral gavage at doses of 0.025, 0.25 and 2.5 mg/kg/day did not result in any mortality, organ weight changes, or macroscopic findings at study completion. In addition, administration of SEQ ID NO 2 to male and female rats at 2.5 mg/kg/day did not result in any microscopic findings at study completion. A small number of animals (1 to 3) among both males and females showed evidence of alopecia on the head and eschar on the face; however, this was not related to the dose of SEQ ID NO 2 and was therefore considered to be incidental in nature and not toxicologically relevant. On Day 89, one control Group 1 female (Animal 7016) had chorioretinal scarring in the right eye. These findings were not attributed to exposure to the test substance; thus, the test substance was not considered an ocular toxicant. All other animals included in the study were normal upon ophthalmic exam. SEQ ID NO 2, up to 2.5 mg/kg/day to male and female rats was tolerable based on hematology, coagulation, clinical chemistry, urinalysis, and thyroid hormone analysis. Increases of TSH in Group 3 (24.5%) and 4 (21.2%) males as well as Group 3 (36.1%) and 4 (38.7%) females may be attributed to the test substance, however there were no changes in T4 and T3 as well as no microscopic correlation despite the ˜2-fold increase in thyroid weight observed in Group 4 males. All other observed statistically significant changes lacked a dose relationship and were comparable to the historical control ranges. Under the conditions of the study and based on the toxicological endpoints evaluated, the no adverse-effect level (NOAEL) for SEQ ID NO 2, administered orally over at least 90 days, was determined to be 2.5 mg/kg/day (the highest dose tested), for both male and female Sprague Dawley rats.

### ADDITIONAL ASPECTS

Aspect 1. A method of increasing telomerase activity and/or increasing telomere length in a population of somatic cells, comprising contacting the population of somatic cells with (1) a polypeptide having at least 80% sequence identity to SEQ ID NO 1 or 2, (2) a polypeptide having at least 85% sequence identity to SEQ ID NO 1 or 2, (3) a polypeptide having at least 90% sequence identity to SEQ ID NO 1 or 2, (4) a polypeptide having at least 95% sequence identity to SEQ ID NO 1 or 2, (5) a polypeptide having at least 98% sequence identity to SEQ ID NO 1 or 2; (6) a polypeptide having at least 99% sequence identity to SEQ ID NO 1 or 2; or (7) a polypeptide according to SEQ ID NO 1 or 2.

Aspect 2. The method of Aspect 1, wherein the population of somatic cells comprises endothelial cells, keratinocyte cells, intestinal epithelial cells, pancreatic cells, pneumonocyte cells, or a combination thereof.

Aspect 3. The method of any one of Aspects 1-2, wherein the method is an ex vivo method and the contacting comprises introducing the polypeptide into the medium in which the population of somatic cells is being cultured.

Aspect 4. The method of any one of Aspects 1-3, wherein the contacting comprises administration of the polypeptide to a mammal comprising the population of cells.

Aspect 5. The method of any one of Aspects 1-4, wherein the contacting comprises oral administration of the polypeptide to a human comprising the population of cells.

Aspect 6. The method of any one of Aspects 1-5, wherein the polypeptide is provided as an oral dosage form.

Aspect 7. The method of any one of Aspects 5-6, wherein the somatic cells are mammalian somatic cells and wherein the contacting comprises oral administration of at least about 1 μg/kg of the polypeptide to the mammal.

Aspect 8. The method of any one of Aspects 5-6, wherein the contacting comprises oral administration of about 30 μg to about 1000 μg of the polypeptide once daily to a human comprising the population of cells, including about 50 μg, about 100 μg, about 200 μg, about 300 μg, about 400 μg, about 500 μg, about 600 μg, about 700 μg, about 800 μg, and about 900 μg of the polypeptide once daily to a human.

Aspect 9. The method of any one of Aspects 1-8, wherein the method comprises increasing telomere length in the population of cells by at least about 10 kilobases per diploid genome, optionally by at least about 20 kilobases per diploid genome.

Aspect 10. The method of any one of Aspects 1-9, wherein the method increases the proportion of progenitor cells in the population of cells by at least about 5% of the population of cells.

Aspect 11. A method of increasing telomerase activity in a cell or tissue, comprising identifying a cell or tissue in which an increase in telomerase activity is desired, and contacting the cell or tissue with (1) a polypeptide having at least 80% sequence identity to SEQ ID NO 1 or 2, (2) a polypeptide having at least 85% sequence identity to SEQ ID NO 1 or 2, (3) a polypeptide having at least 90% sequence identity to SEQ ID NO 1 or 2, (4) a polypeptide having at least 95% sequence identity to SEQ ID NO 1 or 2, (5) a polypeptide having at least 98% sequence identity to SEQ ID NO 1 or 2; (6) a polypeptide having at least 99% sequence identity to SEQ ID NO 1 or 2; (7) a polypeptide according to SEQ ID NO 1 or 2; or (8) fragment(s) thereof.

Aspect 12. The method of Aspect 1, wherein the cell is selected from an endothelial cell, a keratinocyte cell, an intestinal epithelial cell, a pancreatic cell, and a pneumonocyte cell; or wherein the tissue comprises cells selected from endothelial cells, keratinocyte cells, intestinal epithelial cells, pancreatic cells, and pneumonocyte cells.

Aspect 13. The method of any one of Aspects 11-12, wherein the polypeptide has the amino acid sequence set forth in SEQ ID NO: 1; or wherein the polypeptide has the amino acid sequence set forth in SEQ ID NO: 2.

Aspect 14. The method of any one of Aspects 11-13, wherein the method is an ex vivo method and the contacting comprises introducing the polypeptide into the medium in which the cell or tissue is being cultured.

Aspect 15. The method of any one of Aspects 11-14, wherein the contacting comprises administration of the polypeptide to a mammal comprising the cell or tissue.

Aspect 16. The method of any one of Aspects 11-15, wherein the administration comprises an oral dosage form.

Aspect 17. The method of any one of Aspects 15-16, wherein the cells are mammalian somatic cells and wherein the contacting comprises oral administration of at least about 1 μg/kg of the polypeptide to the mammal.

Aspect 18. The method of any one of Aspects 15-16, wherein the contacting comprises oral administration of about 30 μg to about 1000 μg of the polypeptide to a human comprising the cells, including about 50 μg, about 100 μg, about 200 μg, about 300 μg, about 400 μg, about 500 μg, about 600 μg, about 700 μg, about 800 μg, and about 900 μg of the polypeptide once daily to a human.

Aspect 19. A nutraceutical formulation comprising a polypeptide and one or more nutritional carrier;
wherein the polypeptide comprises at least 80% sequence identity to an amino acid sequence set out in SEQ ID NO 1 or SEQ ID NO 2 or fragment(s) thereof; and
wherein the nutraceutical formulation is an aqueous composition, a solid composition, a or a semi-solid composition.

Aspect 20. The nutraceutical formulation of Aspect 19, wherein the polypeptide has at least 95% sequence identity to the amino acid sequence set forth in SEQ ID NO: 1, 98% sequence identity to the amino acid sequence set forth in SEQ ID NO: 1, 99% sequence identity to the amino acid sequence set forth in SEQ ID NO: 1, or comprises or consists of the amino acid sequence set forth in SEQ ID NO: 1; or
wherein the polypeptide has at least 95% sequence identity to the amino acid sequence set forth in SEQ ID NO: 2, 98% sequence identity to the amino acid sequence set forth in SEQ ID NO: 2, 99% sequence identity to the amino acid sequence set forth in SEQ ID NO: 2, or comprises or consists of the amino acid sequence set forth in SEQ ID NO: 2.

Aspect 21. A polypeptide for use in the treatment of an aging-related disease;
wherein the polypeptide comprises at least 80% sequence identity to an amino acid sequence set out in SEQ ID NO 1 or SEQ ID NO 2, or fragment(s) thereof.

Aspect 22. The polypeptide of Aspect 21, wherein the aging related disease is selected from inflammation and joint pain, or irritable bowel syndrome.

Aspect 23. The polypeptide of any one of Aspects 21-22, wherein the polypeptide has at least 95% sequence identity to the amino acid sequence set forth in SEQ ID NO: 1, 98% sequence identity to the amino acid sequence set forth in SEQ ID NO: 1, 99% sequence identity to the amino acid sequence set forth in SEQ ID NO: 1, or comprises or consists of the amino acid sequence set forth in SEQ ID NO: 1; or
wherein the polypeptide has at least 95% sequence identity to the amino acid sequence set forth in SEQ ID NO: 2, 98% sequence identity to the amino acid sequence set forth in SEQ ID NO: 2, 99% sequence identity to the amino acid sequence set forth in SEQ ID NO: 2, or comprises or consists of the amino acid sequence set forth in SEQ ID NO: 2.

Aspect 24. The polypeptide of any one of Aspects 21-23, wherein the contacting comprises administration of the polypeptide to a mammal.

Aspect 25. The polypeptide of any one of Aspects 21-24, wherein the administration comprises a daily dosage of not more than about 200 μg per kg of the mammal, including not more than about 100 μg per kg of the mammal, not more than about 50 μg per kg of the mammal, not more than about 25 μg per kg of the mammal, and not more than about 15 μg per kg of the mammal.

Aspect 26. The polypeptide of any of Aspects 21-25, wherein the polypeptide or composition is provided as an oral dosage form.

Aspect 27. Use (in vivo or ex vivo) of a polypeptide to increase telomerase activity and/or increase telomere length in a population of somatic cells;
wherein the polypeptide comprises at least 80% sequence identity to an amino acid sequence set out in SEQ ID NO 1 or SEQ ID NO 2, or fragment(s) thereof.

Aspect 28. A dietary supplement for human ingestion comprising a polypeptide according to SEQ ID NO 1 or SEQ ID NO 2.

Aspect 29. The dietary supplement of Aspect 28, wherein the supplement is in the form of a liquid, a solid, or a semi-solid.

Aspect 30. A GRAS food product for human ingestion comprising a polypeptide according to SEQ ID NO 1 or SEQ ID NO 2.

Aspect 31. The GRAS food product of Aspect 30, wherein the food product is in the form of a liquid, a solid (e.g., a powder), or a semi-solid.

Aspect 32. An isolated polypeptide according to SEQ ID NO 2.

Aspect 33. An isolated polypeptide according to SEQ ID NO 1 comprising a N- or C-terminal histidine (H) segment (—(H)n-), wherein n ranges from 2 to 10.

Aspect 34. An isolated polypeptide according to SEQ ID NO 2 for use in the manufacture of a dietary supplement or nutraceutical composition.

Aspect 35. The polypeptide of Aspect 21, wherein the polypeptide is provided as an oral dosage form of not more than about 200 μg per kg of the mammal, including not more than about 100 μg per kg of the mammal, not more than about 50 μg per kg of the mammal, not more than about 25 μg per kg of the mammal, and not more than about 15 μg per kg of the mammal.

Aspect 36. The polypeptide of Aspect 21, wherein the polypeptide is provided as an oral dosage form to the mammal in an amount of about 30 μg to about 1000 μg of the polypeptide once daily to a human comprising the population of cells, including about 50 μg, about 100 μg, about 200 μg, about 300 μg, about 400 μg, about 500 μg, about 600 μg, about 700 μg, about 800 μg, and about 900 μg of the polypeptide once daily to a human.

Aspect 37. A composition comprising a polypeptide according to SEQ ID NO 2.

Although the foregoing information highlights aspects disclosed herein by way of illustration and example for purposes of clarity of understanding, it will be obvious that certain changes and modifications may be practiced within the scope of the subject matter claimed herein. It will be clear to a person skilled in the art that features described in relation to any of the aspects and various embodiments described above can be applicable interchangeably between the different embodiments.

The aspects and embodiments described above are examples to illustrate various features of the subject matter claimed herein. All publications and patent applications disclosed herein are indicative of the level of those skilled in the art to which this disclosure and the subject matter of the claims pertains.

Throughout the description and claims of this specification, the words “comprise” and “contain” and variations of them mean “including but not limited to”, and they are not intended to (and do not) exclude other moieties, additives, components, or steps. In certain instances, the expression “comprising” (or its equivalent) may be replaced with the expression “consisting of” (or its equivalent). Throughout the description and claims of this specification, the singular encompasses the plural unless the context otherwise requires. In particular, where the indefinite article is used, the specification is to be understood as contemplating plurality as well as singularity, unless the context requires otherwise.

Features, characteristics, compounds, chemical moieties, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The subject matter claimed herein is not restricted to the details of any foregoing embodiments. The subject matter claimed herein extends to any novel one, or any novel combination, of the features disclosed in this specification (including any accompanying claims, abstract and drawings), or to any novel one, or any novel combination, of the steps of any method or process so disclosed.

All publications and patent applications are herein incorporated by reference to the same extent as if each individual publication or patent application was specifically and individually to be incorporated by reference.

1. A method of increasing telomerase activity and/or increasing telomere length in a population of somatic cells, comprising contacting the population of somatic cells with (1) a polypeptide having at least 80% sequence identity to SEQ ID NO 1 or 2, (2) a polypeptide having at least 85% sequence identity to SEQ ID NO 1 or 2, (3) a polypeptide having at least 90% sequence identity to SEQ ID NO 1 or 2, (4) a polypeptide having at least 95% sequence identity to SEQ ID NO 1 or 2, (5) a polypeptide having at least 98% sequence identity to SEQ ID NO 1 or 2; (6) a polypeptide having at least 99% sequence identity to SEQ ID NO 1 or 2; or (7) a polypeptide according to SEQ ID NO 1 or 2.

2. The method of claim 1, wherein the population of somatic cells comprises endothelial cells, keratinocyte cells, intestinal epithelial cells, pancreatic cells, pneumonocyte cells, or a combination thereof.

3. The method of claim 1, wherein the method is an ex vivo method and the contacting comprises introducing the polypeptide into the medium in which the population of somatic cells is being cultured.

4. The method of claim 1, wherein the contacting comprises administration of the polypeptide to a mammal comprising the population of cells.

5. The method of claim 1, wherein the contacting comprises oral administration of the polypeptide to a human comprising the population of cells.

6. The method of claim 1, wherein the polypeptide is provided as an oral dosage form.

7. The method of claim 1, wherein the somatic cells are mammalian somatic cells and wherein the contacting comprises oral administration of at least about 1 μg/kg of the polypeptide to the mammal.

8. The method of claim 1, wherein the contacting comprises oral administration of about 30 μg to about 1000 μg of the polypeptide once daily to a human comprising the population of cells.

9. The method of claim 1, wherein the method comprises increasing telomere length in the population of cells by at least about 10 kilobases per diploid genome, optionally by at least about 20 kilobases per diploid genome.

10. The method of claim 1, wherein the method increases the proportion of progenitor cells in the population of cells by at least about 5% of the population of cells.

11. A method of increasing telomerase activity in a cell or tissue, comprising identifying a cell or tissue in which an increase in telomerase activity is desired, and contacting the cell or tissue with (1) a polypeptide having at least 80% sequence identity to SEQ ID NO 1 or 2, (2) a polypeptide having at least 85% sequence identity to SEQ ID NO 1 or 2, (3) a polypeptide having at least 90% sequence identity to SEQ ID NO 1 or 2, (4) a polypeptide having at least 95% sequence identity to SEQ ID NO 1 or 2, (5) a polypeptide having at least 98% sequence identity to SEQ ID NO 1 or 2; (6) a polypeptide having at least 99% sequence identity to SEQ ID NO 1 or 2; (7) a polypeptide according to SEQ ID NO 1 or 2; or (8) fragment(s) thereof.

12. The method of claim 11, wherein the cell is selected from an endothelial cell, a keratinocyte cell, an intestinal epithelial cell, a pancreatic cell, and a pneumonocyte cell; or
wherein the tissue comprises cells selected from endothelial cells, keratinocyte cells, intestinal epithelial cells, pancreatic cells, and pneumonocyte cells.

13. The method of claim 11, wherein the polypeptide has the amino acid sequence set forth in SEQ ID NO: 1; or
wherein the polypeptide has the amino acid sequence set forth in SEQ ID NO: 2.

14. The method of claim 11, wherein the method is an ex vivo method and the contacting comprises introducing the polypeptide into the medium in which the cell or tissue is being cultured.

15. The method of claim 11, wherein the contacting comprises administration of the polypeptide to a mammal comprising the cell or tissue.

16. The method of claim 11, wherein the contacting comprises oral administration of about 30 μg to about 1000 μg of the polypeptide to a human comprising the cells.

17. An isolated polypeptide according to SEQ ID NO 2.

18. An isolated polypeptide according to SEQ ID NO 1 comprising a N- or C-terminal histidine (H) segment (—(H)n-), wherein n ranges from 2 to 10.

19. A composition comprising a polypeptide according to SEQ ID NO 2.
